(19) United States
(12) Patent Application Publication
(10) Pub. No.: US 2006/0276818 A1
(43) Pub. Date: Dec. 7, 2006

(54) Safety-stop device

(76) Inventors: John Buser, San Diego, CA (US); Patrick Diesfeld, Ventura, CA (US)

Correspondence Address: FRANK G MORKUNAS, 7750 DAGGET ST, SUITE 203, SAN DIEGO, CA, 92111, US

(21) Appl. No.: 11/146,655

(22) Filed: Jun. 6, 2005

(51) Int. Cl. A61B 17/34

(52) U.S. Cl. 606/185

(57) ABSTRACT

A safety-stop device for use with a trochar which is adjustably attachable to the trochar tube to prevent inadvertent cuts being made to a patient. The safety-stop device has retention component for retaining it onto the trochar tube; a registration component for registering a pre-determined insertion point of the trochar; and a stop component for preventing additional insertion of the trochar after the pre-determined insertion point has been attained.

### CROSS REFERENCES TO RELATED APPLICATIONS

None

### STATEMENT REGARDING FEDERALLY-SPONSORED RESEARCH OR DEVELOPMENT

Not applicable

### BACKGROUND

This safety-stop device relates to an improvement in safety features for trochar assemblies [also referred to as trochar], and more particularly to a safety-stop, attachable to a trochar, registerable on a trochar, and use on a trochar to permit puncture-movement of the trochar only to a pre-determined and pre-set depth.

A trochar is sharp-pointed surgical instrument basically comprised of a stylet [the sharp cutting blades] and a cannula [a small tube for insertion into a body cavity or into a duct or vessel; also referred to herein as trochar tube or a sleeve] and is typically utilized to puncture a body cavity. The stylet is removably housed in the cannula and after the body cavity is punctured, the stylet is removed leaving the cannula in place and in communication with the body cavity whereby endoscopic, and similar, instruments can then be inserted through the cannula and into the body cavity.

Specific forms of minimally invasive surgical procedures include endoscopic and laparoscopic surgery which typically involve using small incisions and optical instrumentation being inserted into the body cavity. Endoscopy refers to video-assisted surgery that is performed through several small incisions rather than a single large incision. Laparoscopy is endoscopy that is done in the abdominal cavity.

The primary instrument used for the incisions necessary in these procedures is the trochar. The use of the trochar for these procedures greatly reduces the amount of cutting required in the course of surgery and, concomitantly, reduction of stress to the body. Reduction of stress to the body generally leads to faster recoveries and lower medical costs. Great care, however, must be exercised when performing such body cavity punctures with the trochar as the trochar blades are ‘surgically’ sharp and the exertion of manual force is required for the blades to pierce and go through the skin and abdominal wall of the patient.

A primary purpose of our safety-stop device is to prevent the sharp trochar blades from accidentally being inserted too deeply. The inherent act of inserting the trochar and attempting to place it where desired requires applying a great deal of initial force down toward the deep anatomic structures, without being able to visualize them, then by sensing a loss of resistance, discontinuing the thrust.

All of this is generally done within a fraction of a second. Patient tissue-variability, in thickness and strength, further complicates the estimation of how much force is required, and for how long, to attain safe penetration.

Excess force, however minimal, or unforeseen factors within the body cavity could lead to piercing or cutting internal organs or other internal structures which could lead to inadvertent and severe life-threatening surgical complications. It has been known that some surgeons extend and use their finger, placed along the sleeve of the trochar, as a makeshift stop. This is awkward, inaccurate, and dangerous.

Trochars manufactured with shielded tips covering the blades; i.e., a safety shield, provide some aid in preventing inadvertent second cuts. While shielded trochar systems vary in their design, all generally have a spring-loaded retractable shield that covers the cutting tip on the blades of the trochar. The shields are either retracted prior to placement of the trochar in the wound or automatically retract during the placement. Once the sharp tip of the trochar's blades penetrates an abdominal wall and enters the abdominal cavity, the spring-loaded safety shield automatically deploys, covering the cutting tip and locking in place.

Theoretically, this prevents or decreases the incidence of damage to the bowel and the major vessels. Injuries can still occur, however, if the trochar is not used properly, if there is a malfunction of the safety shield, or with the presence of bowel adhesions to the anterior abdominal wall. Even with this improvement to the trochar, insertion of the primary trochar blades still remains a blind procedure.

Laparoscopy is a very commonly performed procedure throughout the world. In the U.S. alone, some 6 million cases are performed annually. The total number of cases is growing, as more specialties (general surgery, urology, gynecology) convert procedures over to the laparoscopic approach. Notwithstanding the safety features developed over the years for the trochar, laparoscopy has a background serious complication rate of approximately three to five per 1,000, due to trochar placement. These include intestinal damage, bladder damage, and most seriously large blood vessel (vascular) injury. Of the vascular injuries, which stand at one to two per 1,000, approximately 23% will die.

The majority of serious injuries occur when the stylet of the trochar, with cannula attached, is inserted too deeply, damaging the deeper structures within the body. Only 5 cm. maximum length is necessary, to enter the peritoneal cavity at the umbilicus, the most common entry site. However, trochars are 12 to 15 cm. in length, as a one size -fits-all device. The deep structures, most significantly major blood vessels, can be damaged at 7 to 10 cm., depending on the size of the patient, the degree of gas insufflation raising the abdominal wall, and the angle of thrust executed by the health-care provider.

Our safety-stop device will function to reduce injuries and deaths to patients undergoing laparoscopic surgery. Moreover, our safety-stop device can be made of any material though, for cost considerations, any form of plastic is best suited. Furthermore it can, but need not, be disposable for further patient safety as a one-time use.

Some unique features of our safety-stop device include:

a. shortening the effective length of the dangerous sharp trochar/sleeve, by gripping the outer sleeve, with a wider diameter cuff;

b. it can be used with any of the major trochar/sleeve manufacturers' current product-lines;

c. it does not impede appropriate surgical trochar/sleeve entry, only inadvertent deep entry;

d. it does not impede performance of the remainder of the surgery;

e. it is compatible with any other “safety-features” built into other areas of the trochar/sleeve;

f. it can be placed anywhere along the sleeve, to shorten the effective length, at the surgeon's discretion, for that particular-sized patient;

g. it does not require abandoning current surgeon preferences of trochar/sleeve;

h. it does not require new or further training to use;

I. it will be inexpensive to manufacture and will not thereby add significant cost to a surgical case;

j. it can, and will, save lives; and

k. it will save the health-care system millions of dollars in patient disability, emergency rescue surgery, and malpractice exposure.

The foregoing has outlined some of the more pertinent objects of the safety-stop device. These objects should be construed to be merely illustrative of some of the more prominent features and applications of the safety-stop device. Many other beneficial results can be attained by applying the disclosed safety-stop device in a different manner or by modifying the safety-stop device within the scope of the disclosure.

Accordingly, other objects and a fuller understanding of the safety-stop device may be had by referring to the summary of the safety-stop device and the detailed description of the preferred embodiment in addition to the scope of the safety-stop device defined by the claims taken in conjunction with the accompanying drawings.

### SUMMARY

The above-noted problems, among others, are overcome by the safety-stop device. Briefly stated, the safety-stop device contemplates a device for use with a trochar and which is adjustably attachable to the trochar tube to prevent inadvertent cuts being made to a patient. The safety-stop device has retention means for retaining it onto the trochar tube; a registration means for registering a pre-determined insertion point of the trochar; and a stop means for preventing additional insertion of the trochar after the pre-determined insertion point has been attained.

The foregoing has outlined the more pertinent and important features of the safety-stop device in order that the detailed description that follows may be better understood so the present contributions to the art may be more fully appreciated. Additional features of the safety-stop device will be described hereinafter which form the subject of the claims. It should be appreciated by those skilled in the art that the conception and the disclosed specific embodiment may be readily utilized as a basis for modifying or designing other structures and methods for carrying out the same purposes of the safety-stop device. It also should be realized by those skilled in the art that such equivalent constructions and methods do not depart from the spirit and scope of the safety-stop device as set forth in the appended claims.

### BRIEF DESCRIPTION OF THE DRAWINGS

For a fuller understanding of the nature and objects of the safety-stop device, reference should be had to the following detailed description taken in conjunction with the accompanying drawings in which:

FIG. 1 is perspective view of a typical trochar assembly.

FIG. 2 is an elevation, cross-sectional, exploded view of a preferred embodiment of the safety-stop device.

FIG. 3 is a plan view of the cap component of the safety-stop device as taken on line 3-3 of FIG. 1.

FIG. 4 is a cross-section view of the opposite side, rotated 180°, of the insert as illustrated in FIG. 2.

FIG. 5 is a plan view of the insert as taken on line 5-5 of FIG. 4.

FIG. 6 is an elevation view of a second embodiment of the safety-stop device.

FIG. 7 is a cross-section elevation view of the cap illustrated in FIG. 6.

FIG. 8 is a plan view of the base of the safety-stop device as taken on line 8-8 of FIG. 6.

FIG. 9 is a perspective exploded view of a third embodiment of the safety-stop device.

### DETAILED DESCRIPTION

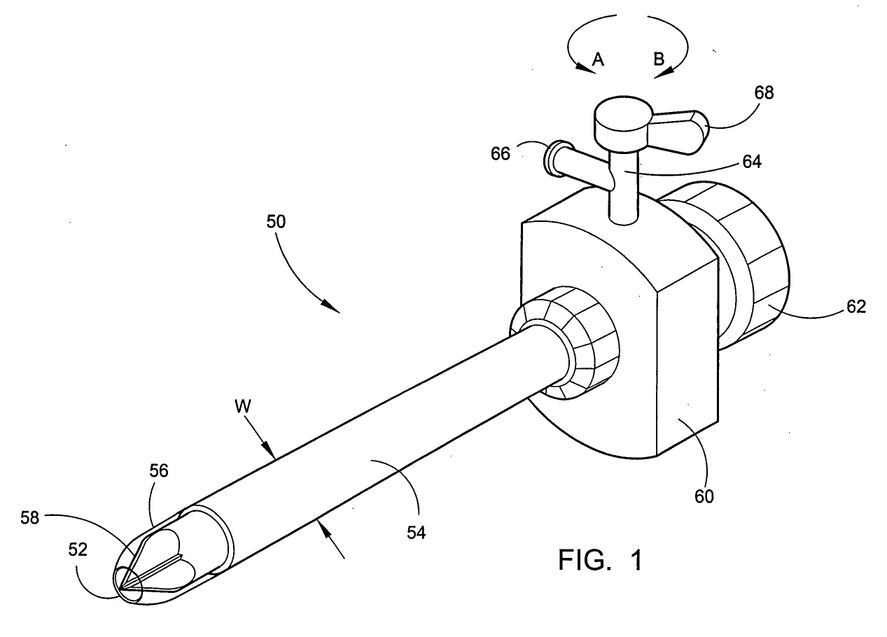

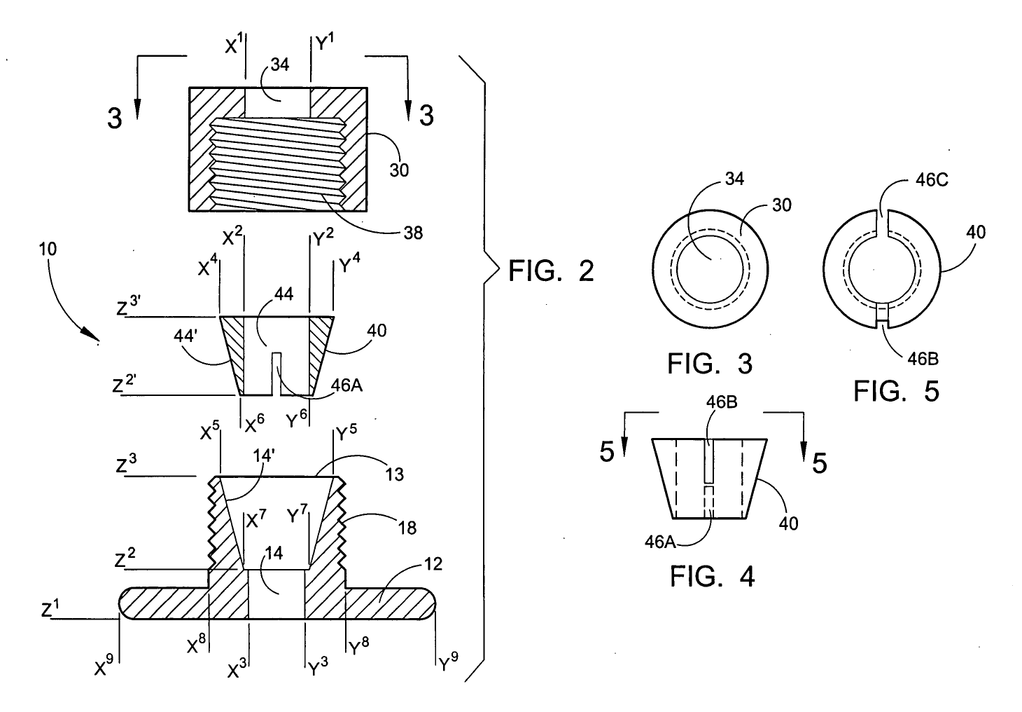

Referring now to the drawings in detail and in particular to FIG. 1, a typical trochar assembly 50, and reference character 10 of FIG. 2 which generally designates a safety-stop device constructed in accordance with a preferred embodiment thereof.

The trochar assembly 50 has a set of blades 58 attached to a knob 62 which, after the trochar assembly 50 is used, is removable from the cannula 54 [trochar tube]. The cannula 54, having a width W, is attached to a head assembly 60 with its components [reference characters 64, 66, 68] thereon. A blade shield 56 with an shield opening 52 at its far end covers the blades 58 to facilitate safe handling of the trochar assembly 50 and to prevent accidental cuttings.

Typically once the trochar assembly 50 has been used to execute the surgical procedure, the surgeon grasps the knob 62 and pulls the blades 58 with blade shield 56 out from the cannula 54. The cannula 54 remains and is in communication with the body cavity into which the trochar assembly 50 penetrated. Suitable hoses [not shown], for example, are connected to the inlet/outlet 66 on valve 64, and the valve lever 66 is positioned on/off [arrows A, B] to, for example, force air or gas into the patient as, and if, necessary to the procedure.

The safety-stop device 10 has a base component 12, an insert component 40, and a cap component 30. The base 12 has a stem 13 [upstanding member] attached thereto and a base channel 14 which is bore completely therethrough from the bottom (point Z1] of the base to and out of the top of the upstanding member 13 [point Z3].

The width of the channel 14 at the bottom of the base is X3-Y3. The width at the opening on the top of the upstanding member 13 is X5-Y5. Width X5-Y5 is greater than width X3-Y3 in that the channel 14 initiates an outward angling 14′ above the bottom of the base 14 at approximately point Z2 which bears a width X7-Y7 and terminates at the top [point Z3] of the upstanding member 13 defining an opening thereat having width X5-Y5 wherein width X5-Y5 is greater than width X7-Y7 and wherein width X7-Y7 could be equal to or greater than width X3-Y3. The width of the base 12 [X9-Y9] is substantially greater than the width of the upstanding member 13 [X8-Y8]. It is this base width [X9-Y9] which functions as a stop. The external surface of upstanding member 13 is threaded.

The insert 40, a collet-like component, is configured to fit and seat into the base channel 14, 14′ at approximately point Z2 with its exterior surface 44′ bearing an angle approximately equal to angle 14′. In this regard, the insert 40 has a height [Z2′ to Z3′] which is slightly larger than the distance from point Z2 to point Z3. The insert 40 has a bore 44 vertically disposed therethrough [insert channel]. The width of the insert channel 44 is X2-Y2. The width of the top of the insert 40 is X4-Y4 and the width at the bottom of the insert 40 is X6-Y6. As the insert 40 is structured to seat into the base channel 14, width X4-Y4 is slightly greater than width X5-Y5, and width X6-Y6 is slightly greater than width X7-Y7.

As illustrated in FIGS. 2 and 4, the insert 40 may have one or more vertical slots 46A on the bottom [FIG. 2] or one or more vertical slots 46B on the top or any combinations thereof. The slots may be extend upward or downward, respectively, partially or, as illustrated in FIG. 5, vertically extend the full distance from top to bottom [reference character 46C].

The cap 30 has a hollow interior with threading 38 on the interior surface thereof. The interior threading [female threading] 38 of the cap 30 corresponds with the exterior threading 18 [male threading] of the upstanding member 13. Once the insert 40 is seated into the upstanding member 13 and the cap 30 threaded over the upstanding member 13 a sealing, retaining, and registering unit is formed. On the top of the cap 30 is a cap aperture 34 which has a width X1-Y1.

In this embodiment widths X1-Y1, X2-Y2, X3-Y3 are approximately equal and each are approximately equal to or slightly greater than the trochar tube 56, width W. In operation, there are many methods of attaching the safety-stop device 10 to the trochar assembly 50; i.e., whether the trochar assembly 50 is inserted into the safety-stop device 10 after the safety-stop device 10 has been assembled as a unit or before such assembly as a unit, or whether the assembled or unassembled safety-stop device 10 is inserted onto the trochar assembly 50, or any combinations there.

The final configuration will have the cap 30 distal from the shield opening 52 with the threaded interior 38 facing the shield opening 52. Next is the insert 40, wider end first, followed by the base 12 with its bottom facing the shield opening 52. The insert 40 is seated into the upstanding member 13 and the cap 30 and the upstanding member 13 are connected. In view of the larger size of the insert 40, this connection causes the insert 40 to press against the cannula 54 and tighten and secure around it. The slots 46A, 46B, 46C, depending on which configuration of insert 40 is being used, are squeezed and close or pinch in the process. The tighter the connection between cap 30 and base/upstanding member unit 12, 13, the more securely the safety-stop device 10 is contained on the cannula 54.

Loosening the connection between the base/upstanding member unit 12, 13, loosens the connection between the safety-stop device 10 and the cannula 54 to thereby permit the safety-stop device 10 to translate back and forth on the cannula 54 to any desired point for a pre-determined depth in execution. Once that pre-determined point is established, the safety-stop device 10 is secured to the cannula 54, the trochar assembly is ready to use, and the base 12 acts as a stop once it contacts the skin of the patient to prevent further penetration into the patient's body cavity.

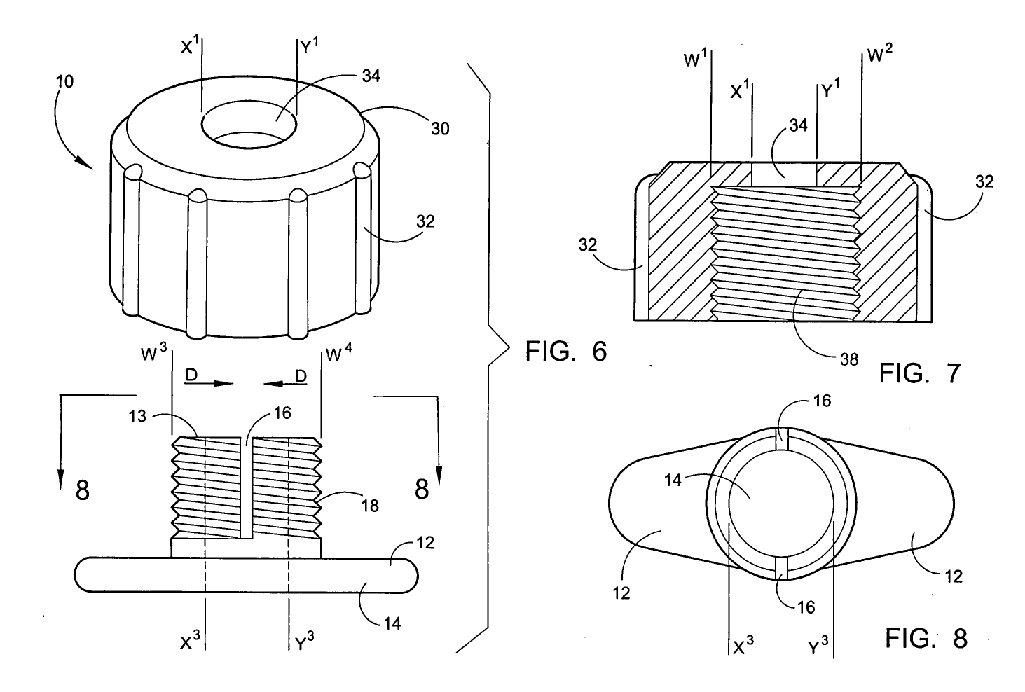

FIGS. 6-8 illustrate a slightly different safety-stop device 10 configuration without an insert 40. Here the base 12 and upstanding member 13 unit are configured externally basically as described above. In this embodiment the upstanding member 13 has one or more vertically disposed slots 16, no internally angled walls 14′, and a width W3-W4 at the top. The cap 30 is basically identical except that is has one or more vertically disposed fins 32 on its exterior surface to aid the user in tightening the cap 30 onto the base/upstanding member unit 12, 13. The threading 38 on the inside surface is somewhat tapered in that the width W1-W2 at the top is less than width W3-W4 at the top of the upstanding member 13.

The cap aperture 34 has a width X1-Y1 and the channel aperture 14 at the bottom of the base 12 has a width X3-Y3. Each of these widths [X1-Y1 and X3-Y3] are equal to or slightly greater than the trochar tube 54 width W.

As before, once the safety-stop device 10 and the trochar assembly 50 are attached, tightening the cap 34 over the upstanding member 13, with its smaller width W1-W2, squeezes the upstanding member 13 tightly [in the directions of Arrows D] over the trochar tube 54. Loosening the cap 34 permits the user to slide the safety-stop device 10 to any desired location on the trochar tube 54, re-tighten the cap, and use the trochar assembly 50.

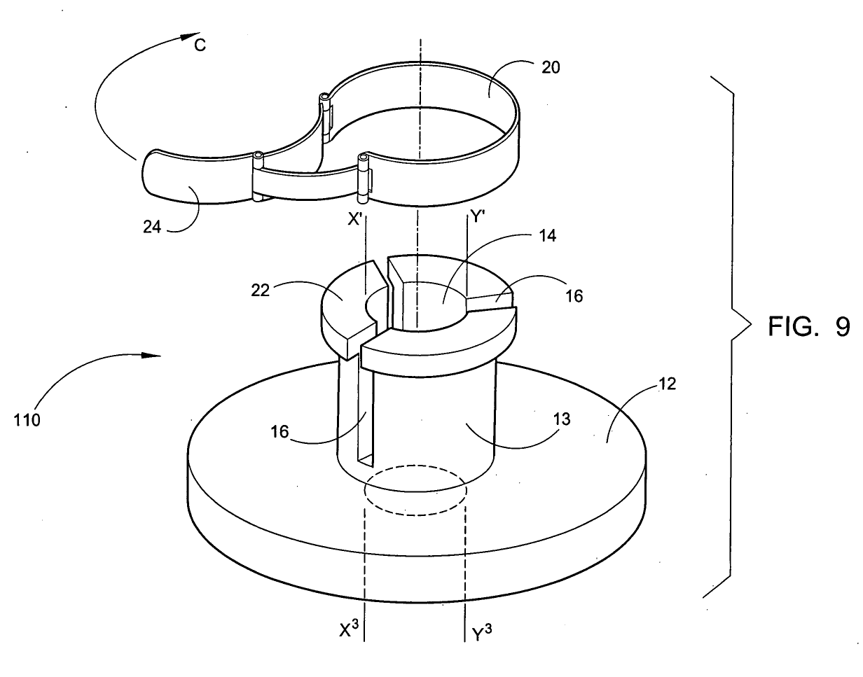

A third embodiment of the safety-stop-device 110 is illustrated I FIG. 9. Here the upstanding member 13 on the base 13 is not threaded. It has one or more vertically disposed slots 16 and may, but need not, have a collar 22 at the top of the upstanding member 13 to aid in retaining the clamp 20. In operation, the clamp 20 generally is first placed on the trochar tube 54 followed by the base/upstanding member unit 12, 13 through the base channel 14.

Once the unit 12, 13 is slid on the trochar tube 54 where desired, the clamp 20 is placed over the upstanding member 13 and secured thereover. Any suitable clamping device will suffice. As illustrated in FIG. 9, an over-center clamp 20, with lever 24, is utilized because of its ease of use to lock and unlock the clamp 20. Simply moving the lever 24 in the direction of Arrow C tightens the clamp 20 over the upstanding member 13, and squeezes the upstanding member 13 tightly on the trochar tube 54. The slots 16 in the upstanding member 13 cause the upstanding member 13 to be more flexible and, with the pressure of the clamp 20, cause the upstanding member 13, as with the slots described in the previous embodiment, to tightly hold the trochar tube 54.

The base 12 in any embodiment may be round, as illustrated in FIG. 9, or may have one or more side wings, as illustrated in FIG. 8, or may bear any geometric shape suitable for the intended purpose; i.e., to be a stop member. The safety-stop device 10 may be made of any suitable materials, including, but not limited to plastics.

The present disclosure includes that contained in the present claims as well as that of the foregoing description. Although this safety-stop device has been described in its preferred forms with a certain degree of particularity, it is understood that the present disclosure of the preferred forms has been made only by way of example and numerous changes in the details of construction and combination and arrangement of parts and method steps may be resorted to without departing from the spirit and scope of the safety-stop device. Accordingly, the scope of the safety-stop device should be determined not by the embodiments illustrated, but by the appended claims and their legal equivalents.

What is claimed is:

1. A safety-stop device for use with a trochar having a trochar tube, said device comprising:
(a) retention means for retaining said device onto said trochar tube;
(b) registration means for registering a pre-determined insertion point of said trochar; and
(c) stop means for preventing additional insertion of said trochar after said pre-determined insertion point has been attained.

2. A safety-stop device for use with a trochar having a trochar tube, said device comprising:
(a) a base with a bottom and an upstanding member on said base wherein said base has a channel extending from said bottom through a top of said upstanding member wherein said base further has a width substantially greater than a width of said upstanding member; and
(b) a cap having a hollow interior, a cap aperture, and retention means for retaining said cap onto said upstanding member after placement thereon thereby defining a unit and for registering and retaining said unit on said trochar tube after placement thereon.

3. The device as claimed in claim 2 wherein said retention means further comprises external threading on said upstanding member and cooperating threading on said hollow interior of said cap.

4. The device as claimed in claim 3 wherein said cap further comprises one or more lateral outward extensions on its exterior surface.

5. The device as claimed in claim 2 wherein said channel has a channel width, said cap aperture has a aperture width, and said trochar tube has a tube width each of which are approximately equal in width to one another.

6. The device as claimed in claim 5 wherein said upstanding member further comprises one or more vertical slots.

7. The device as claimed in claim 5 wherein said channel is angled outward thereby defining an opening at the top of said upstanding member wherein said opening is greater than said channel width.

8. The device as claimed in claim 7 wherein said retention means further comprises an insert adapted to seat into said upstanding member, said insert having an insert channel disposed therethrough with an insert channel width approximately equal to said trochar tube width.

9. The device as claimed in claim 8 wherein said insert further comprises one or more vertically disposed slots.

10. The device as claimed in claim 5 wherein said upstanding member further comprises a collar on top of said upstanding member, wherein said collar has a width greater than said width of said upstanding member.

11. The device as claimed in claim 10 wherein said upstanding member further comprises one or more vertically disposed slots extending upward through said collar.

12. The device as claimed in claim 10 wherein said retention means comprises a clamp adapted to insert onto said upstanding member and be unlockably locked thereon.
